(19) United States
(12) Patent Application Publication
(10) Pub. No.: US 2015/0007856 A1
(43) Pub. Date: Jan. 8, 2015

(54) METHOD FOR TREATING A SUBSTRATE SURFACE USING OZONATED SOLVENT AND ULTRAVIOLET LIGHT

(71) Applicant: David Jackson, Saugus, CA (US)

(72) Inventor: David Jackson, Saugus, CA (US)

(21) Appl. No.: 14/325,278

(22) Filed: Jul. 7, 2014

Related U.S. Application Data
(60) Provisional application No. 61/843,730, filed on Jul. 8, 2013.

(51) Int. Cl. B08B 7/00; B08B 3/08

(52) U.S. Cl. CPC B08B 7/0057; B08B 3/08
USPC 134/1

(57) ABSTRACT

A method and an apparatus for removing or modifying a portion of a substrate surface, including contaminants thereon, using volatile methyl siloxanes (VMS) treatment fluid containing a controlled level of dissolved ozone (O₃) gas and ultraviolet (UV) light is provided.

### PRIORITY CLAIM

This application claims the benefit of U.S. Provisional Patent Application 61/843,730 (filed 8 Jul. 2013), which is incorporated by reference in entirety.

### BACKGROUND OF INVENTION

The present invention relates to the field of precision cleaning and surface treatment. More particularly, the present invention is a novel hybrid cleaning process that eliminates the constraints of conventional organic solvent cleaning, ozonated solvent cleaning, or ultraviolet (UV)-ozone cleaning. The present invention is illustrated in an example with regard to removing thick hard varnish and carbon deposits from a fuel injector nozzle, including a method and apparatus, but it will be recognized that the invention has a wider range of applicability. Merely by way of example, the invention can also be applied to the manufacture of ophthalmic lenses (cleaning and etching for coating), electronic flip chip devices (defluxing), medical devices (bioburden reduction), processing wafers (photoresist residue removal), washing biomimetic textiles (extraction and disinfection), and removing fingerprints and particles from electronic devices (cosmetic cleaning), and many other applications.

Much interest exists to develop alternative dry cleaning and treatment methods to replace hazardous chemicals such as organic solvents, acids and hydrogen peroxide. Moreover, the desire to eliminate the use of precious water resources as cleaning and rinsing agents in manufacturing is becoming a socioeconomic imperative. Technological advances in manufacturing and materials result in more complex assemblies and features, with multiple materials compatibility constraints, and more numerous multiple cleaning and contamination control challenges. For example in microelectromechanical systems (MEMS), shrinking line widths and trenches with high aspect ratios require advanced cleaning and drying technologies. Industry utilizes or has proposed various techniques to remove organic photoresists and particles, rinse and dry a semiconductor wafer.

Ozone is an attractive cleaning agent and additive. Ozone is a naturally occurring triatomic form of oxygen which, under atmospheric temperature and pressure, is an unstable gas that decomposes readily into molecular oxygen. With an electrochemical oxidation potential of 2.07, ozone is a powerful oxidant and is used commercially in precision cleaning systems in combination with deionized water. Ozone can be generated in-situ and following use, decomposes back into oxygen gas. The ozone molecule is slightly polar, with a dipole moment of 0.53 D. As such, ozone is only slightly soluble in water but is highly soluble in non-polar organic solvents such as carbon dioxide, fluorocarbons, and carbon tetrachloride, possessing cohesion energy between approximately 15 MPa^{1/2} and 20 MPa^{1/2}.

The ozone molecule dissolved in a solvent acts upon the contaminant or native organic substrate surface by direct or indirect oxidation, by ozonation or catalysis. The three major reaction pathways occur as follows:
Direct oxidation reactions of supercritical ozone, resulting from the action of an atom of oxygen, are typical first order high redox potential reactions
Indirect oxidation reactions of ozone, the ozone molecule decomposes to form free radicals (i.e., OH ions, peroxides) which quickly oxidize organic and inorganic compounds
Ozone may also act by ozonolysis, that is, by fixing the complete molecule on the double linked atoms (double bonds), producing two simple molecules with differing properties and molecular characteristics

However, many ozone reactions and reaction rates can be very slow or near-zero, particularly involving low concentrations of ozone gas and low temperature and complex contaminants or films. Ultraviolet light is known catalyst for ozone reactions. As such, an aspect of the present invention is to accelerate these reactions either within the treatment fluid and/or at the substrate surface containing the contaminant or film to be treated. In addition, it is known that sonication accelerates ozone reactions, and as such is used with the present invention for same as well as to adjust ozone levels within the treatment fluid. For example using the present invention, the treatment bath containing the substrates is sonicated while the reacted treatment fluid is removed, treated with UV light, filtered, and recirculated back into the treatment bath.

Ozone-loaded solvents or ozonated solvents including ozonated water, ozonated fluorocarbons, ozonated esters and ozonated acids have been developed to address various cleaning challenges over the past 20 years. Ozonated cleaning solvents offer advantages over prior art methods such as strong organic solvents and acids, but are not without their own constraints. These are discussed further as follows.

With respect to cleaning wafers to remove photoresist residue contamination, commercial cleaning systems have been developed which employ ozone and water to replace dangerous or ecologically-unsafe chemical processes such as sulfuric acid-hydrogen peroxide mixtures, toxic organic solvents, and amine-based cleaning agents. One such system, called the SMS DIO3 photoresist strip process (Legacy Systems Inc., Fremont, Calif.), uses an ozone generator and diffuser located in a tank of chilled (5 degrees C.) deionized water, to increase ozone concentration, which is circulated into a tank containing the wafers. Water has a cohesion parameter of 47.3 MPa^{1/2} and a dipole moment of approximately 3 D, compared to the ozone cohesion parameter of approximately 17 MPa^{1/2} and dipole moment of 0.53 D. In addition, the surface tension of water is very high—72.8 dynes/cm at 25 degrees C. As such, the ozone molecule exhibits only slight solubility in water and wets only a very small portion of an organic surface—contaminant or technical surface. Moreover, the aforementioned system suffers from an inability to apply thermal energy to the substrate because it lowers the solubility of ozone in solution even more. As such, the DIO3 process is a highly time-dependent and concentration-dependent cleaning process.

Another commercialized process, called HydrOzone (trademark of Semitool Inc.), diffuses ozone gas through a thin film of heated water which is spreading over a fast spinning wafer—both of which increases the water-solid contact area (i.e., lowers surface tension) and accelerates ozone reactions through mechanical and thermal actions. The HydrOzone process is claimed to be more efficient than the DIO3 process above because the water component may be heated to provide thermal cleaning energy and revolutions-per-minute (RPM)) may be varied to control boundary layer thickness. However, similar to the DIO3 process, transport of ozone of any significant concentration into micron features on the wafer surface is very limited due to the still relatively high surface tension of heated ozonated water—for example 67.9 dynes/cm at 50 degrees C.

Other ozone-loaded solvent cleaning processes have been developed to both increase ozone solubility (ozone concentration) and contaminant solubility to improve cleaning power. These have generally employed organic solvents and acidified water.

Exemplary prior art in this regard include U.S. Pat. No. 6,537,380 (also referred to as '380) issued to Zazerra et al; U.S. Pat. No. 6,696,228 (also referred to as '228) issued to Muraoka et al; and U.S. Pat. No. 6,867,148 (also referred to as '148) issued to Yates et al. In U.S. Pat. No. '380, a non-polar fluorinated solvent is ozonated and used to clean a contaminated surface. The fluorocarbon solvent has a cohesion parameter of approximately 15 MPa^{1/2}, a surface tension of approximately 15 dynes/cm, and a dipole moment of 0 D. Although inert, non-toxic, and have an ability to dissolve large amounts of ozone gas, the principal drawback of ozonated fluorocarbons is their high cost and low boiling points. As such, ozonated fluorocarbons require closed systems for economical use. In U.S. Pat. No. '228, an ozonated polar ester (i.e., hot propylene carbonate) is used to first dissolve photoresist from a patterned wafer, following which the dissolved photoresist contained within the cooled solvent is partially decomposed with ozone gas. Although a strong solvent for dissolving photoresist, particularly at higher temperatures, the main drawback of using polar ester solvents is its low ozone solubility particularly at higher temperatures, similar to the challenges of creating high dosages of dissolved ozone gas in water. For example, propylene carbonate has a relatively high cohesion parameter of 27 MPa^{1/2} and a dipole moment of 4.9 D, compared to the ozone cohesion parameter of approximately 17 MPa^{1/2} and dipole moment of 0.53 D. The process of '228 relies on the solvent power of the ester with the ozone cleaning actions predominantly on the dissolved resist contaminant. Finally, in U.S. Pat. No. '148, ozonated and acidified water is used to increase solubility of ozone and to provide metal passivation to protect the substrate from the potential corrosive effects of aqueous ozonated treatment.

Finally, another conventional hybrid cleaning process related to the present invention utilizes ultraviolet (UV) light and ozone gas conducted within a non-solvent fluid atmosphere such as air or nitrogen. Exemplary and foundational prior art for the UV-ozone cleaning method is provided in U.S. Pat. No. 3,664,899, issued to Wright, and assigned to General Electric Co., and U.S. Pat. No. 3,914,836, issued to Hafner et al, and assigned to the U.S., Secretary of Army. The UV-ozone cleaning procedure is an effective method of rapidly removing a variety of contaminants from surfaces. UV-ozone treatment is a very simple-to-use and relatively low-cost dry cleaning process which is inexpensive to set up and operate. It can produce clean surfaces in air and at ambient temperatures, relying on the significant energy provided by ions, radicals and ionizing radiation. A variety of contaminants can be successfully removed from substrate surfaces, including oils and greases (including silicones), fluxes, skin oils, and organic contamination adsorbed during prolonged exposure to air. Experiments performed with low-pressure mercury discharge UV sources, one which generates ozone and one which does not, demonstrates that the combination of short-wave UV light plus ozone gas produces a clean surface substantially faster than either short-wave UV light without ozone or ozone without UV light. However there are many constraints for this process. The most important variables for this conventional technique are the level of contamination initially present on the surface, the necessity of a pre-cleaning procedure, the wavelengths emitted by the UV source, the nature of the atmosphere between the UV source and sample, the distance between the UV source and sample, and the time of exposure. Surfaces must be properly pre-cleaned to remove gross contamination and placed within a few millimeters of an ozone-producing UV source to consistently produce a clean surface very quickly (within several minutes). Also as the atmosphere between the UV source and substrate surface changes, the cleaning rate changes substantially due to absorption of UV light by oxygen and ozone or the lack of ozone gas completely. For example, nitrogen gas purging and atmospheric dilution can be utilized to increase UV source to substrate distance, however the drawback is a decrease in ozone concentration and subsequent cleaning rate. Another drawback is line-of-sight limitations. As such, the UV-ozone cleaning technique is useful mainly for cleaning simple substrate geometries such as lenses, optics, ceramics and other UV-resistant and simple geometries. Conventional UV-ozone cleaning has been useful only for very low production of a select group of contaminated substrate applications. A more detailed description of the UV-ozone cleaning process can be found in “UV/Ozone Cleaning of Surfaces”, Vig, J., Journal of Vacuum Science and Technology, May/June 1985.

As the complexity of manufactured hardware increases, it is clearly desirable to have a non-aqueous processing technique, including a method and apparatus, that removes unwanted organic films ad particles, prevents additional particles, and does not introduce stains on the surfaces. The complete surface treatment technique may also include a step of increasing the surface energy and modifying or functionalizing the same in preparation for adhesive bonding, coating, and other operations.

From the above, it is seen that a method and apparatus for precision cleaning and treating substrate surfaces that is safe, easy, and reliable is desired. As such there is a present need to provide an alternative advanced oxidative cleaning and surface treatment process, apparatus and method which overcome the limitations of conventional ozonated chemistry and UV-ozone cleaning technology.

### SUMMARY OF THE INVENTION

A method and an apparatus for removing or modifying a portion of a substrate surface, including contaminants thereon, using volatile methyl siloxanes (VMS) treatment fluid containing a controlled level of dissolved ozone (O₃) gas and ultraviolet (UV) light is provided. VMS is a very safe, low cost and abundantly available compound, and offers unique advantages in the present invention including minimal absorption and reactivity with ultraviolet (UV) light and ozone gas, very low surface tension, good hydrocarbon solubility, excellent substrate compatibility, and biocompatibility. VMS containing dissolved ozone is contacted with an organic surface portion (technical surface and/or contaminant surface) thereon to be etched, removed and/or functionalized. The contacted surface and/or ozonated VMS is exposed to UV light to catalytically assist with removal of surface contaminants and/or etch and/or activation of treated substrate surface, and the breakdown of dissolved organics contained within the ozonated VMS, respectively. Moreover, conventional cleaning energy adjuncts such as ultrasonics are employed to adjust ozonation levels and to accelerate cleaning actions. A spectrophotometric device is used to monitor ozone and dissolved contamination levels within the VMS treatment fluid. Finally, VMS treatment fluid exhibits very high solubility in carbon dioxide (CO₂), allowing it to be rinsed and recovered from treated substrate surfaces and purified and reused using environmentally safe CO₂ spray and immersion rinsing and recovery techniques.

The present invention provides a method and apparatus for precision cleaning and treating substrate surfaces (organic or inorganic surfaces) which may contain upon it, or within it if it is porous or fibrous, a wide range of organic and inorganic contaminations or films. The present invention uses a unique silicon-based ozonated treatment fluid—a biocompatible, material compatible, UV-transmissive, ozone-unreactive, low-energy, and high-boiling organic solvent called volatile methyl siloxanes or VMS—in cooperation with ultraviolet (UV) light as well as sonication. The treatment fluid of the present invention exhibits hydrocarbon-like cleaning action, high ozone gas solubility, and excellent UV light transmission. UV radiation is transmitted within the treatment fluid to catalyze the formation of oxygen radicals from ozone gas and to facilitate ozonation reactions therein to enhance the cleaning effect (removal and decomposition) upon a substrate surface. Contaminants contained on a substrate surface and those solubilized as UV-ozonated by-products within the treatment fluid during treatment processes are removed, dissolved and decomposed, thus allowing the treatment fluid to regenerate for reuse.

An exemplary VMS for use in the present invention is decamethylcyclopentasiloxane (“D5”), also called a Pentamer. VMS (D5) has a very low freeze point, high boiling point, and very low water solubility. As such, VMS (D5) can be used as a low temperature cleaning fluid—facilitating much larger ozone concentrations and very low solvent losses during use—except for thin film evaporative losses from treated substrates. VMS (D5) has a very low cohesion energy which provides high ozone solubility and lower losses. VMS (D5) can be used at a wide range of process temperatures from +100 degrees C. to −20 degrees C. without concern for dissolving atmospheric water into solution. The invention of '228, for example, uses a highly polar solvent that will absorb a significant amount of water vapor if subcooled and if exposed to the atmosphere. Moreover, the invention of '228 cannot be used with ultraviolet light. This is due to the reactivity of the ester-based carrier solvent with UV light and UV-catalyzed ozonation reactions. VMS (D5) is a low-cost alternative to the solvent used in U.S. Pat. No. '380 as well as a recyclable and renewable non-aqueous alternative to the solvent used in U.S. Pat. No. '148. Moreover the present invention can optionally employ sonication to accelerate ozonation reactions as well as to adjust ozonation levels within the treatment fluid.

Among cleaning solvents, VMS (D5) is considered a very weak cleaning solvent because of its low cohesion energy and lack of polarity. VMS (D5) has a cohesion energy value of approximately 16 MPa^{1/2} and possesses a KB value well below 25. More complex surface contaminants such as photoresists, varnishes, biological deposits, cross-linked polymers, and carbon deposits cannot be solubilized, nor significantly swelled, by VMS, even in the presence of heat and sonication. It is nonetheless attractive as a base solvent due to its wide ranging material compatibility, biocompatibility, non-toxicity, non-corrosiveness, low surface tension, and ability to solubilize a wide range of fairly simple organic molecules.

VMS is uniquely suited for the present invention due to low freeze point and high boiling point, its ability to carry high concentrations of ozone gas (due to its low cohesion energy) and ability to transmit ultraviolet light and ultrasound with minimal interaction. Negligible ozone and UV-ozone reaction by-products of VMS include traces of low molecular weight siloxanes, silicon dioxide, water, and hydrated silica (silicon hydroxide) which are further reacted and filtered from solution along with contaminant degradation by-products. Moreover, because of its very low surface tension of 18.5 dynes/cm and low viscosity of 3.87 cst, ozonated D5 wets a larger surface area of a substrate to contact higher concentrations of ozone against larger portions of a contaminant or film more quickly, thus providing higher flux in the cleaning process. The cohesion parameter of D5 is in the range of between approximately 15 MPa^{1/2} and 20 MPa^{1/2}, ideal for the cohesion energy range of ozone and ozonated by-products of contaminants such as carboxylic acids, esters, and ketones. As such, the exemplary VMS (D5) as used in the present invention serves as a temperature- and ozone-adjustable treatment media for facilitating ozone-only and UV-ozone treatment reactions and providing adequate solvency for low molecular weight by-products of sonication, ozonolysis, and/or UV-ozonolysis.

For example varnish build-up within diesel fuel injector nozzles is a result of free-radical chain reactions within the diesel fuel generated by hot combustion processes. These reactions comprise chain initiation, propagation, branching, and termination steps, all of which produce successive layers of resinous fuel deposits over time that eventually constrain fuel flow. Such contamination must be removed from the nozzles periodically to return them to as-new fuel flow condition. Strong solvents with ultrasonics must be used to dissolve the varnish due to its nature and complexity—a highly cross-linked, plastic-like and gummy surface coating. Acidic cleaner are not useful for this application as they will corrode the carbon steel along with the contaminant.

In another example, biological endotoxin (BET) is a chemical contaminant derived from the cellular wall of biological cells. It is a highly polar lipid complex containing 5 moles of acyl, 5 moles of ether, 2 moles of amine, 2 moles of organophosphate, and 4 moles of alcohol functional groups. This molecule is a portion of the lipopolysaccharide, called lipid A, and is very toxic to humans. For example if even very small amounts of lipid A contaminate an implantable medical device surface, it poses a significant threat of a dangerous internal inflammatory response to patients in contact with same. Sterilization techniques are ineffective and aqueous or solvent washing cannot be relied upon to remove this contaminant. The solubility parameter for the lipopolysaccharide or LPS is 33 MPa^{1/2}. As such simple hydrocarbon solvents such as fluorocarbons, and including VMS (D5), cannot solubilize this compound, nor denature the lipid A molecule to mitigate human toxic reaction if exposed. Complex surface geometry such as porosity further constrains BET cleaning processes. Typically, strong acids and thermal treatments are used to denature or decompose the BET contaminant in place. However, these treatment methods are not practical for many medical devices due to material and process compatibility challenges.

In still another example, organometallic photoresist residues remain across a wafer surface following development and processing steps to produce read-write heads for hard disk drives. This residue must be removed in successive steps during production. Ozonated water, ozonated solvents, acid etchants and/or vacuum plasma treatments are used as conventional cleaning methods, with the various drawbacks discussed herein. For example if UV light is used with ozonated water, this would introduce significant corrosion issues due to the presence of water and the formation of corrosive water-based acids. As such, it is desirable to have a safer, faster and lower cost alternative to conventional ozone-based photoresist residue removal methods.

Finally, exemplary VMS (D5) used in the present invention, and as compared to the prior art, is neither water soluble nor completely volatile. It cannot be rinsed from treated surfaces using water and following air drying, the surface will contain (or entrap) a monolayer or more of VMS (D5). As such if it is desirable to remove residual VMS (D5) from treated substrates, the preferred method of solvent rinsing and recovery of VMS (D5) is environmentally friendly CO₂ centrifugal liquid immersion and/or composite spray rinsing processes developed by the present inventor, and discussed in detail in U.S. Pat. No. 6,802,961 and U.S. Pat. No. 7,451,941, respectively. Other techniques such as heated air blow-off or vacuum drying may also be employed.

The present invention overcomes the constraints of conventional cleaning applications such as those discussed above. UV-catalyzed ozonolysis is used to produce simpler and lower molecular weight compounds such as carboxylic acids, ketones and aldehydes—derived from the more complex surface contaminants (i.e., varnish, BET and photoresist) contained on a substrate surface. As a result, the UV-ozone reacted and lower molecular weight by-products having lower cohesion energies readily soluble within VMS (D5). Upon dissolution, these simpler contaminant by-product molecules are further oxidized to nitrogen, carbon dioxide and water—discharged from the treatment media into the atmosphere or absorbed onto filtration media. Dissolved salts and solvent-insoluble metals released during cleaning are typically precipitated as solids and filtered from the VMS (D5) process bath. Using this novel approach, both substrate surface and treatment fluid are decontaminated, providing a close-loop cleaning and treatment fluid regeneration process.

In summary, the present invention manipulates the reaction environment using ozone and UV-catalyzed ozone as the primary reactant in combination with VMS as benign cleaning and carrier agent. The entire process can be controlled using ozone concentration, UV light intensity and exposure, treatment fluid temperature and cleaning adjuncts such as ultrasonics to aid in ozonolysis and UV-ozonolysis reactions and cleaning rates. A UV-VIS spectrophotometer is used to conveniently monitor the dissolved ozone levels and VMS-solubilized organic by-product levels to control the process and determine the quality of the treatment fluid. Finally, adjunct CO₂ immersion and spray rinse treatments developed by the present inventor may be used to remove and recover the VMS fluids from treated surfaces for reuse in the present invention.

A first aspect of the present invention provides a method for removing an organic contaminant or film on a surface of a substrate, comprising: bringing a highly UV-transmissive and UV-unreactive treatment liquid comprising liquid volatile methyl siloxanes (VMS), into contact with said substrate to contact said organic contaminant or film, thereby allowing ozone gas and UV light or ultrasound waves to interact intimately with ozone diffusing into and reacting with said organic contaminant or film, and allowing the low molecular weight reaction by-products to migrate into said treatment liquid, decomposing said material further in said treatment liquid to even lower molecular weight material by UV-ozonolytic pathways, thereby said UV-ozone treated treatment liquid being regenerated as a treatment liquid, and recycling the treatment liquid thus regenerated for treating another substrate.

A second aspect of the present invention provides a method for removing an organic contaminant or film on a surface of a substrate, comprising: rinsing and recovering treatment liquid from treated substrate by rinsing same with a dense fluid such as a CO₂ Composite Spray™ (Trademark of CleanLogix LLC) or Centrifugal CO₂™ immersion (Trademark of CleanLogix LLC).

In a preferred embodiment of the second aspect of the present invention, said treatment liquid after removal of said organic contaminant or film is recycled as a treatment liquid as it is for treating another substrate.

In another preferred embodiment of the second aspect of the present invention, said treatment liquid after removal of said organic contaminant or film is further subjected to treatment with UV-ozone, and then recycled as a treatment liquid for treating another substrate.

A third aspect of the present invention provides an apparatus for removing an organic contaminant or film from a surface of a substrate comprising: (A) a treatment liquid delivery means for transporting a treatment liquid comprising liquid VMS to a substrate treatment area, (B) a contaminant or film contact means for bringing the treatment liquid into contact with the surface of said organic contaminant or film of the substrate within the treatment area, (C) a treatment liquid circulation means for recycling treatment liquid used and discharged from the treatment area back to said treatment area via one or more temporary storage means, and (D) an ozone-containing gas and UV light contact means for bringing an ozone-containing gas and UV light into contact with the treatment liquid discharged from said treatment area within said treatment area and/or within at least one of said temporary storage means.

A fourth aspect of the present invention provides an apparatus for monitoring the levels of dissolved ozone gas and ozone-reacted organic by-products dissolved in the treatment fluid. Because the treatment fluid of present invention is uniquely and highly transparent to radiation in visible range all the way down to the UV, a low-cost UV-VIS spectrophotometer provides detailed information about the chemistry and quality of VMS treatment fluid—measuring ozone gas levels and dissolved organic by-product levels.

### BRIEF DESCRIPTION OF THE DRAWINGS

FIG. 1 illustrates the chemical structure and nature of the treatment liquid VMS, describing the low at various wavelengths of light from near-infrared to ultraviolet.

FIG. 2 is a flowchart illustrating the cleaning method and other aspects of the present invention.

FIG. 3 is a schematic of an apparatus employing the present invention in an immersion method.

FIG. 4 is a schematic of an apparatus employing the present invention in a single substrate spin method.

FIG. 5 illustrates the use of the present invention to remove varnish from a fuel injector nozzle.

### DETAILED DESCRIPTION OF THE PREFERRED EMBODIMENTS

As follows is a more detailed description of the present invention.

### Treatment Liquid

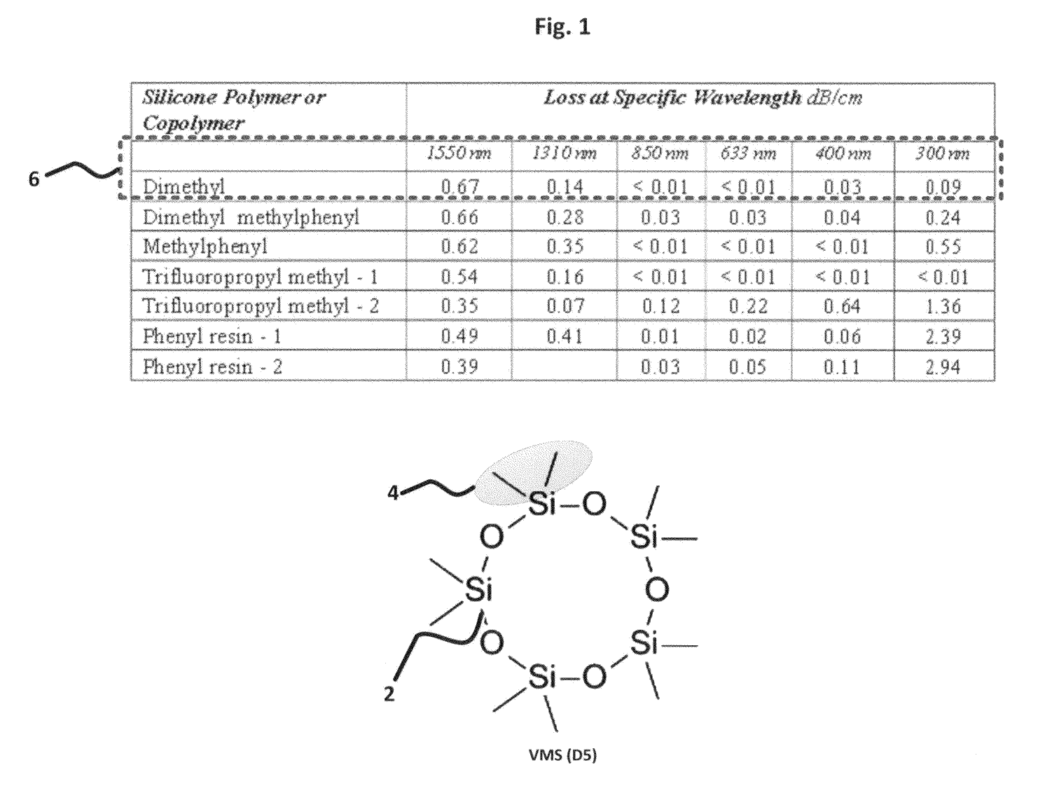

Referring to FIG. 1, the preferred treatment fluid of the present invention is volatile methyl siloxanes—and more preferably decamethylcyclopentasiloxane, D5, or simply VMS (D5)—a cyclic molecule (2). The dimethyl groups (4) comprise the organic nature of the VMS (D5) molecule. An aspect of the present invention is the use of ultraviolet (UV) light to accelerate ozonation reactions to assist removal of surface contaminants or films and/or to accelerate dissolved contaminant decomposition using ozone. As shown in FIG. 1, VMS (D5) exhibits minimal loss (6) in the UV, visible (Vis) and near-infrared (NIR) range from 300 nm to 1550 nm. This optical clarity enables the transmission of UV light for in-situ UV-ozone treatment processes and UV-Vis-NIR light for on-line analytical procedures of the present invention.

VMS solvents are insoluble in water, and at room temperature are stable colorless and odorless liquids. VMS (D5) is a preferred solvent in the present invention due to favorable physical and chemical properties such as high boiling point and flash point, biocompatibility, ozone resistance, UV transmission, and low toxicity. Exemplary VMS and properties are shown under Table 1 and Table 2, respectively.

TABLE 1
Exemplary Volatile Methyl Siloxanes (VMS)

| Chemical Name | INCI Name | Short Name |
|---|---|---|
| Decamethylcyclopentasiloxane | Cyclopentasiloxane | D5 |
| Dodecamethylcyclohexasiloxane | Cyclohexasiloxane | D6 |

TABLE 2
Physical Properties of Exemplary VMS

| Property | D5 | D6 |
|---|---|---|
| Melting Point, ° C. | −38 | −3 |
| Boiling Point, ° C. | 211 | 245 |
| Density, g/cm3 at 25° C. | 0.954 | 0.963 |
| Vapor Pressure, Pa at 25° C. | 33.2 | 4.6 |
| Water Solubility, mg/L at 25° C. | 0.02 | 0.005 |

These VMS liquids exhibit weak solvent action with respect to polar hydrocarbons, due to its low solubility parameter (SP value), between approximately 15 MPa^{1/2} to 16 MPa^{1/2}. However, VMS solvents are non-polar, and consequently the solubility of ozone gas is very high. The ozone incorporated within a VMS displays a powerful decomposition effect on dissolved organic material and particularly compounds with double bond linkages or aromatic compounds—with or without UV. By-products of ozonation processes are more soluble in the VMS solvents.

The solvent power of VMS liquids increases with increasing temperature. Provided the temperature is below the flash point, the treatment operation can be performed safely in open-air systems. Furthermore, the vapor pressure of VMS is fraction of other organic solvents such as for example n-methylpyrollidone (NMP), offering the advantage that liquid loss through evaporation under heating is far lower. At high temperatures, this evaporation loss increases slightly, but the toxicity of the vapor is extremely low, meaning the evaporation is not a significant problem.

During the removal of a contaminant or film on a substrate by a liquid treatment, by irradiating high frequency ultrasound (so-called ultrasonics or megasonics) of a frequency from 40 kHz to 2 MHz through the liquid, substrate damage arising from cavitation can be suppressed, and the chemical action of the liquid, and particularly ozone, is amplified through highly accelerated molecular agitation. By employing this auxiliary mechanical treatment, the cleaning performance of the treatment liquid can be enhanced markedly.

The preferred “treatment fluid” herein comprises VMS (D5) saturated with ozone gas at a temperature of between 5 degrees C. and 100 degrees C. Temperature control is achieved using a suitable liquid chiller and heater, both of which are available from a number of commercial sources. Saturation is achieved by diffusing ozonated air or oxygen into temperature-regulated VMS (D5) fluid for a period of time as necessary to achieve ozone saturation. Ozone saturation can be monitored on-line using a spectrophotometer or off-line using iodometry. When ozone gas is passed through and dissolved in a liquid, the distribution coefficient D is represented by the formula D=C_{L}/C_{G}, where C_{G} [mg/L] represents the ozone concentration in the gas and C_{L} [mg/L] represents the ozone concentration in a saturated liquid. According to the literature, the solubility of ozone in a solvent is greater for non-polar solvents such as carbon tetrachloride, for which the value of D at room temperature is approximately 1.5 to 2. In contrast, the value of D at room temperature for polar solvents is approximately 0.4 at most, and is approximately 0.2 in the case of pure water. Furthermore, regardless of the type of solvent, the value of D decreases as the temperature increases. In other words, ozone solubility decreases with increased temperature.

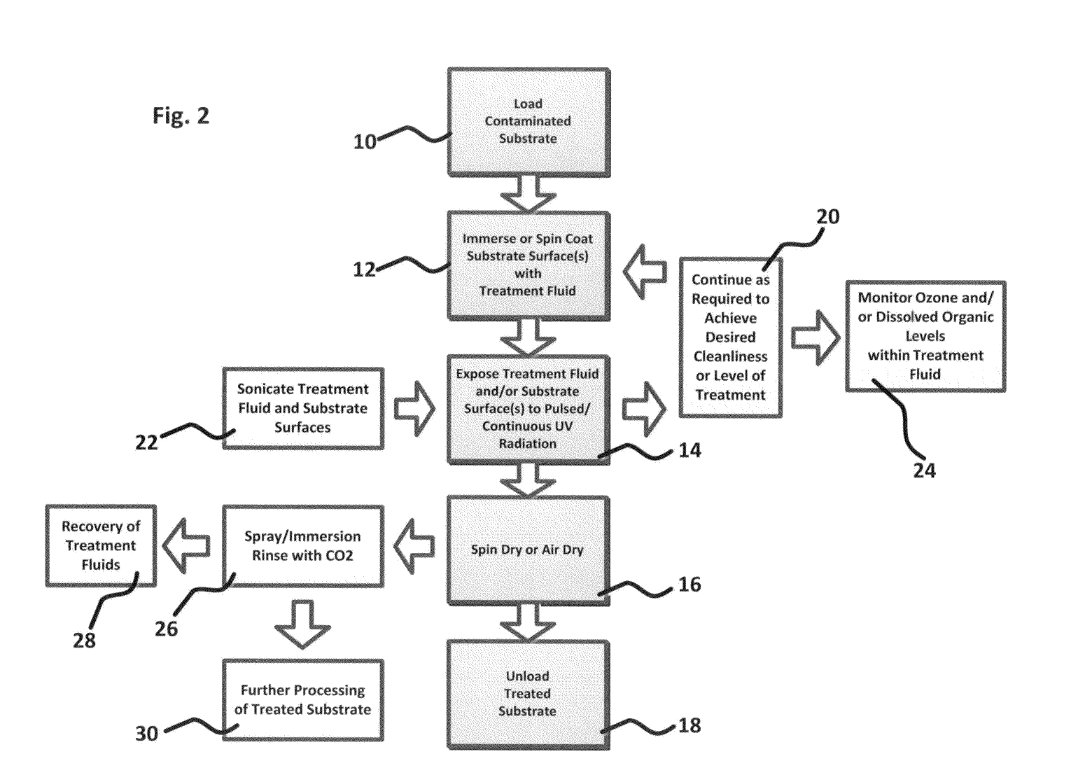

Now referring to FIG. 2, the preferred substrate cleaning or treatment method of the present invention comprises the following steps:
1. Load contaminated substrate into a basket or mount to a spin processer (10);
2. Immerse or spray contaminated substrate with treatment fluid at a temperature of between 5 degrees C. and 100 degrees C., or more, and at a pressure of between 1 atm (immersion) and 4 atm (spray) (12);
3. Expose treatment fluid and/or contaminated substrate surface to a source of pulsed or continuous UV radiation having a wavelength between about 200 nm to 400 nm (14);
4. Drain, air dry or spin dry treated substrates free of residual treatment fluid (16); and
5. Unload cleaned or treated substrates (18).

The entire treatment process comprising steps 2 through 3 is continued (20) as necessary to achieve a pre-determined level of cleanliness or treatment. Cleaning exposure times may vary between 30 seconds and 2 hours, or more. In addition to ozonation and UV-catalyzed ozone treatment, acoustic energy may be employed to enhance ozone reactions and cleaning rates. Acoustic radiation in the range between 40 kHz and 2 MHz may be applied to the treatment fluid and substrate (22) continuously or in pulses during the treatment steps 2 through 3. During cleaning and treatment operations, dissolved ozone and contamination may be monitored in-situ or ex-situ using a variety of analytical techniques (24).

Finally, cleaned and treated surfaces of the present invention may contain residual VMS treatment fluids condensed onto or trapped within pores of the substrate surfaces. This level may not be acceptable for certain follow-on substrate production operations, for example coating or bonding. Although a number of techniques may be employed to remove this residual treatment fluid—including for example hot air blow-off, vacuum extraction and thermal extraction—the preferred precision “rinsing” technique of the present invention is exemplary carbon dioxide (CO₂) based immersion and/or spray processes (26) developed by the present inventor, and examples of which are discussed in detail in U.S. Pat. No. 6,802,961 and U.S. Pat. No. 7,451,941, respectively, and incorporated herein by reference to same. For example, using one or both of these techniques as a precision rinsing step allows for the recovery of the treatment fluid (28) for reuse in treatment steps 2 through 3 above, and the production of precision clean substrates (30) which are ready for follow-on production steps such as patterning, adhesive bonding, machining, welding, coating, assembly and sterilization. As described above, by using an ozonated VMS liquid treatment process, organic contaminants or films can be effectively removed and components derived from the organic film end up dissolved (or dispersed) within the treatment fluid. The treatment fluid containing dissolved organic material is further aerated with ozone gas and exposed to UV light to decompose same into even lower molecular weight contaminants such as gases and water.

Consequently, provided the treatment liquid is aerated with ozone gas and exposed to UV light following substrate treatment, the liquid can be circulated and reused, as is. If there are concerns about residual non-decomposed contaminants such as particles, then microfiltration may be performed if necessary. As such, conducting UV-ozone treatment offers the significant advantage of enabling the treatment liquid to be recycled. This ability to recycle the treatment liquid offers enormous economic benefits when compared with conventional, expensive organic solvent treatments. In the present invention, by using a recycling technique wherein the treatment liquid is treated with UV-ozone and microfiltered, the treatment liquid can be reused dozens of times without requiring replacement with fresh treatment liquid.

In addition, in the present invention, the step for taking the treatment liquid following removal of an organic film and aerating with ozone gas and exposure to UV light to decompose any components derived from the organic film down to low molecular weight materials may be performed using a batch method, and may also be conducted in a different area, such as a different building, from the area in which the organic film removal is performed. In such a case, where necessary a tank lorry may be used for transporting the treatment liquid long distances to the aforementioned different area.

The number of times the liquid can be circulated (in other words, the lifespan of the treatment liquid) will vary depending on the quantity of oxidizing materials generated at each ozone treatment and the purity of the treatment liquid, which will gradually decrease. The quantity of oxidizing materials generated by ozone treatment can be determined in real-time and on-line, or off-line using a UV-Vis-NIR spectrophotometry, for example, or iodometric analytical techniques.

Ozone gas diffusion should be discontinued and the treatment fluid degassed using ultrasonic cavitation destruction or clean air purging. Simply turning off the ozonator and continuing to diffuse clean dry air into the treatment fluid is effective for stripping residual ozone gas from solution.

### Action of Dissolved Ozone (and UV) on Treatment Liquid

As described above, organic film components which migrate into the treatment liquid during the organic film removal treatment are decomposed by ozone or UV-ozone treatment. Consequently, if VMS (D5) containing dissolved ozone is used in the organic film removal process, then the synergistic effect of the solvency action of the treatment liquid solvent together with the decomposition action of the ozone or UV-catalyzed ozone, and ultrasound, results in a markedly improved cleaning performance even for treatment temperatures of 40 degrees C. or lower.

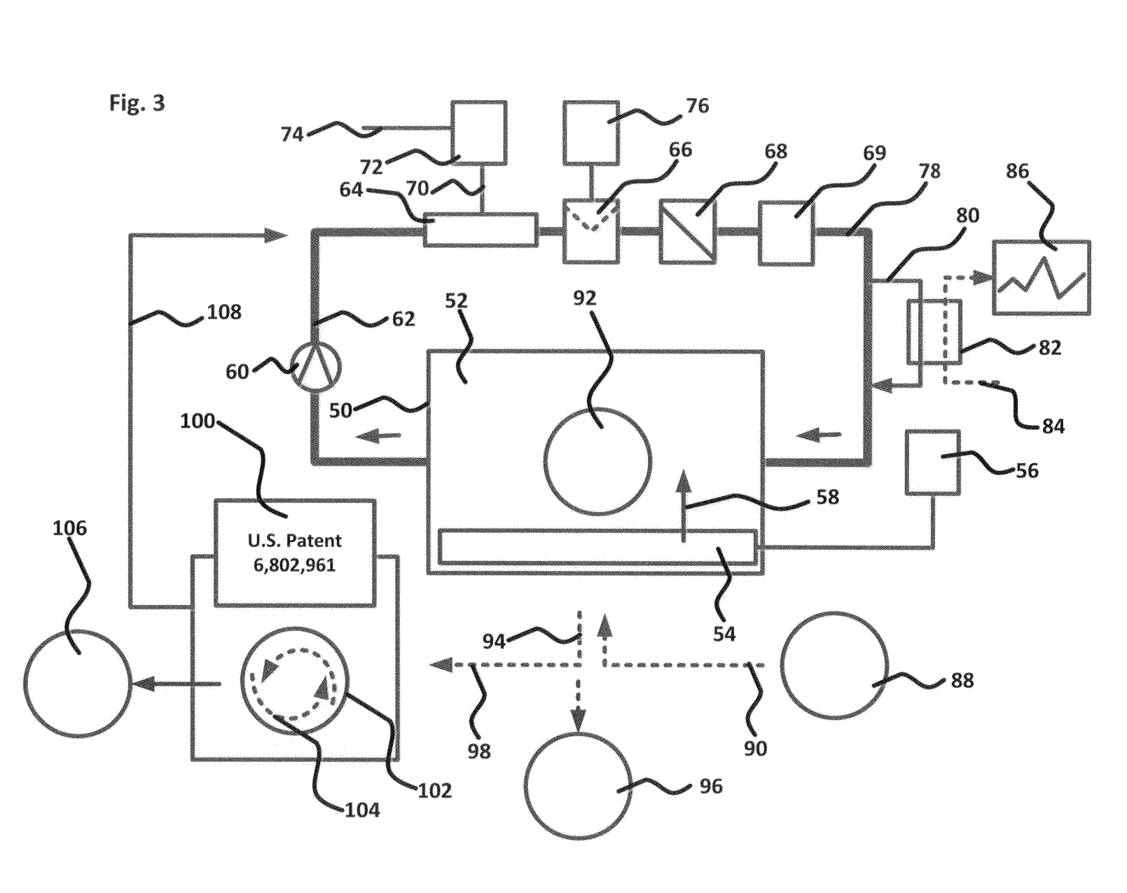

An exemplary immersion treatment system for using the present invention in described under FIG. 3. Referring to FIG. 3, the exemplary system comprises a treatment tank (50) containing treatment fluid (52). The exemplary treatment tank (50) contains a tubular titanium ultrasonic resonator (54) and ultrasonic power supply (56), for example a Model RS-40-40-X-40 kHz system and power supply available from Telsonic Ultrasonics, Germany, to produce either pulsed or continuous acoustic radiation (58) within the treatment fluid (52). Treatment fluid is withdrawn from the treatment tank (50) using a centrifugal pump (60) and piped (62) into a ozonation and treatment train comprising an ozone gas injection and diffusion membrane or static mixer (64), UV light treatment cell (66), microfilter (68), and treatment fluid temperature controller (69). The ozone injector (64) is fluidly connected (70) to an ozone gas generator (72), the exemplary ozone generator and injector system such as the Liquozon® System available from MKS Ozone Products, Andover, Mass., which is connected to a source of pressure-regulated clean dry air or oxygen gas (74). The UV treatment cell (66) is connected to a UV power supply and controller (76). An exemplary UV treatment cell (66) is the Trojan UVMAX E4 Plus UV System, available from numerous water purification equipment providers. A suitable microfilter (68), such as a 100% fluorocarbon Guardian® AT filter element, available from Entegris, is needed for removing residual particles and rejecting free water from the system. A suitable treatment fluid temperature controller (69) is available from a variety of commercial sources. Finally, ozonated, UV-treated, microfiltered, and temperature-controlled treatment fluid is returned (78) back to the treatment tank (50) for reuse.

An exemplary and optional analytical system is also described under FIG. 3. A portion of the ozonated, UV-treated, and microfiltered treatment fluid is piped (80) into a flow cell (82), such as a Model FIA-Z-SMA-PEEK-LENSED from Ocean Optics, which is coupled with a suitable light source (84), such as halogen light, and a spectrophotometer (86), such as for example the STS Miniature Spectrophotometer operating with a wavelength range of between 350-800 nm, available from Ocean Optics. Using this system, and coupled with a computer and software, real-time analysis of dissolved ozone levels and UV-ozone degraded and dissolved organic contamination contained with the treatment fluid can be ascertained.

The use of the exemplary immersion cleaning system of FIG. 3 and method of FIG. 2 is described as follows. Contaminated substrates contained in a suitable basket (88) are transported (90) and immersed (92) within the treatment tank (50) containing said treatment fluid (52) operating at a temperature of between 5 degrees C. and 100 degrees C. The contaminated substrates are subjected to ultrasonic radiation (58) for a pre-determined period of time. Optional UV radiation (not shown) may also be beamed into the treatment fluid, in addition to acoustic radiation or by itself, to expose the contaminated substrates to same. Following treatment, treated substrates are lifted from the treatment tank (50) and treatment fluid (52), drained for a pre-determined period of time, and transported (94) to a off-load station (96). Alternatively, and in accordance with a preferred precision rinsing embodiment of the present invention, treated substrates may be transported (98) to an exemplary centrifugal liquid CO₂ rinsing system and process (100), whereupon treated substrates (102) are subjected to bi-directional centrifugal rinsing processes (104), in accordance with exemplary U.S. Pat. No. 6,802,961, issued to the present inventor. The treated and rinsed substrates (106) are ready for follow-on production steps such as assembly, machining, welding, coating, bonding, and sterilization. Treatment fluid rinsed and recovered from the treated parts (102) using the exemplary centrifugal CO₂ immersion rinse system (100) is piped (108) back to the treatment system for reuse.

In the case of an organic film removal and etch in which the treatment liquid contacts the substrate surface in the form of a moving liquid film using a spin processor, if the treatment liquid is saturated with ozone in an ozone saturation vessel and then supplied to the substrate surface through a nozzle, then similar excellent removal effects to those described above can be achieved. In order to achieve the required ozone concentration, the temperature of the liquid should preferably be not more than 50 degrees C. At high temperatures the ozone concentration decreases significantly, and consequently the piping linking the ozone saturation vessel and the nozzle within the organic contaminant or film removal apparatus should be kept as short as possible.

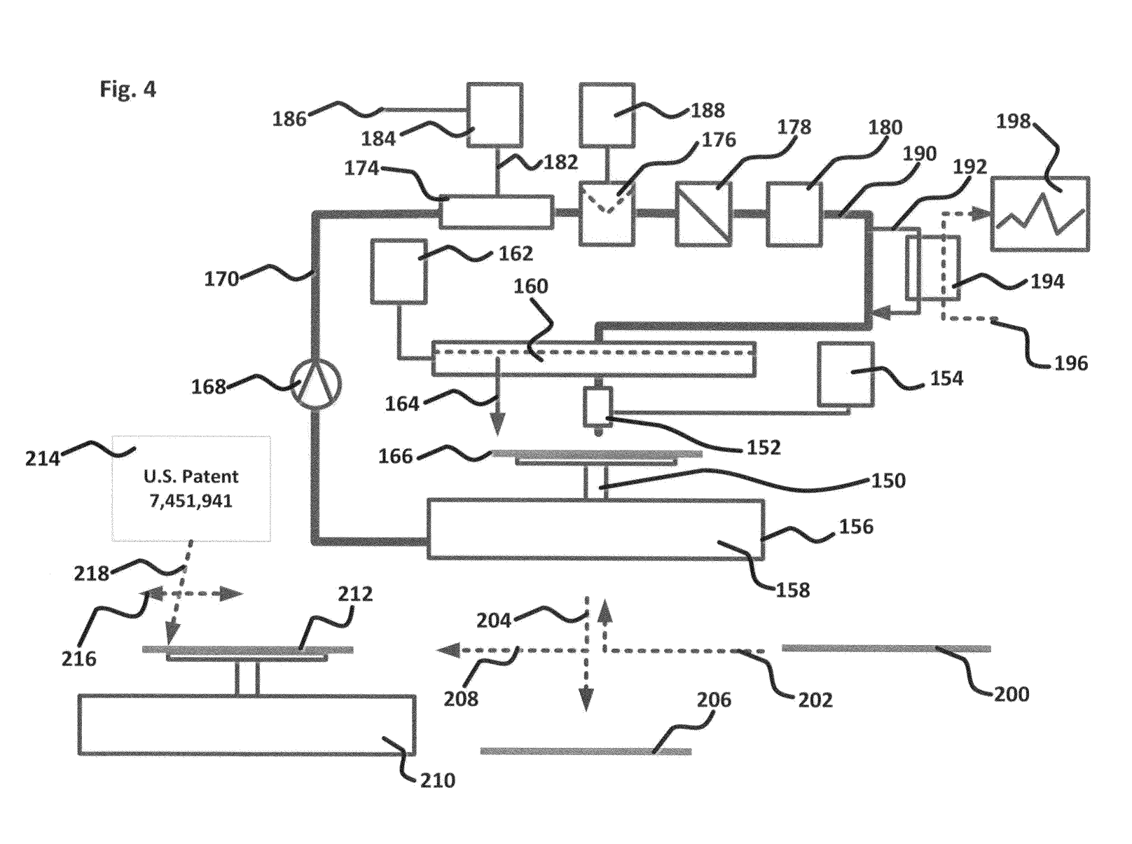

Now referring to FIG. 4, the exemplary system comprises a spin processer (150) containing an acoustic spot spray nozzle (152) and power supply (154), and treatment fluid storage tank (156) for containing treatment fluid (158). For example, if a 430 kHz megasonic spot shower is used as the spray nozzle, such as the Kaijo Model 27200, then the spin cleaning rate can be improved even further. This spin cleaning method is particularly useful if a process requires a powerful cleaning treatment at a temperature of 50 degrees C. or higher, such as in the case of cleaning and etching ophthalmic thermoplastics such as polycarbonate. The exemplary spin cleaning and treating system also uses a UV light source (160) and power supply (162), for example a Pulsed Xenon system from Xenon Corporation, to produce pulsed or continuous UV radiation (164) which is directed at the spin processor (150) and substrate (166) contained thereon. Treatment fluid (158) is withdrawn from the treatment fluid storage tank (156) using a centrifugal pump (168) and piped (170) into a ozonation and treatment train comprising an ozone gas injection and diffusion membrane or static mixer (174), UV light treatment cell (176), microfilter (178), and treatment fluid temperature controller (180). The ozone injector (174) is fluidly connected (182) to an ozone gas generator (184), the exemplary ozone generator and injector system such as the Liquozon® System available from MKS Ozone Products, Andover, Mass., which is connected to a source of pressure-regulated clean dry air or oxygen gas (186). The UV treatment cell (176) is connected to a UV power supply and controller (188). An exemplary UV treatment cell (176) is the Trojan UVMAX E4 Plus UV System, available from numerous water purification equipment providers. A suitable microfilter (178), such as a 100% fluorocarbon Guardian® AT filter element, available from Entegris, is needed for removing residual particles and rejecting free water from the system. A suitable treatment fluid temperature controller (180) is available from a variety of commercial sources. Finally, ozonated, UV-treated, microfiltered, and temperature-controlled treatment fluid is returned (190) and is directed through the spot shower (152) and over the center of the spinning substrate (166). Excess treatment fluid is spun from the substrate and returned to the treatment fluid storage tank (156) for reuse.

An exemplary and optional analytical system is also described under FIG. 4. A portion of the ozonated, UV-treated, microfiltered, and temperature-controlled treatment fluid is piped (192) into a flow cell (194), such as a Model FIA-Z-SMA-PEEK-LENSED from Ocean Optics, which is coupled with a suitable light source (196), such as halogen light, and a spectrophotometer (198), such as for example the STS Miniature Spectrophotometer operating with a wavelength range of between 350-800 nm, available from Ocean Optics. Using this system, and coupled with a computer and software, real-time analysis of dissolved ozone levels and UV-ozone degraded and dissolved organic contamination contained with the treatment fluid can be ascertained.

The use of the exemplary immersion cleaning system of FIG. 4 and method of FIG. 2 is described as follows. A contaminated substrate, for example an ophthalmic polycarbonate lens (200), is transported (202) and mounted to the spin processor (150). The substrate is vacuum held and spun to between 100 and 5000 RPM, during which the substrate is sprayed with treatment fluid (52) having a temperature of between 5 degrees C. and 100 degrees C. The contaminated substrates are subjected to ultrasonic radiation from the spot shower (152) for a pre-determined period of time. UV radiation (164) is beamed onto the spinning substrate. Following treatment, treated substrates (166) are de-spun and lifted from the spin processor (150) and transported (204) to a off-load station (206). Alternatively, and in accordance with a preferred precision rinsing embodiment of the present invention, treated substrates may be transported (208) to an exemplary centrifugal liquid CO₂ spray rinsing system and process (210), whereupon treated substrates (212) are subjected to spray rinsing processes (214), using a CO₂ spray process described under exemplary U.S. Pat. No. 7,451,941, issued to the present inventor. The treated substrate (212) is scan cleaned (216) using the exemplary CO₂ spray (218) while the treated substrate (212) is spun. The treated and precision rinsed substrate is ready for follow-on production steps.

### EXAMPLE OF USE

As follows, an organic contamination and film removal method and apparatus according to the present invention is described. However, the present invention is in no way restricted to the example presented below.

Removing Varnish Build-Up from Fuel Injectors

The ozone gas used in the following example was produced by passing clean dry air-comprising for example −40 degree F. dew point, <10 ppm hydrocarbons, and filtered using a 0.5 micron gas filter—through a commercial ozonator, Portazone Ozone Purification System, Model PZ-250, producing 250 mg ozone per hour, available from Superior Health Products. The VMS-D5-used was of reagent grade purity, available from VMR Scientific or Fischer Scientific, was contained in a temperature-controlled 40 kHz ultrasonic cleaning tank set at 40 degrees C. Prior to commencement of the cleaning process, ozonated gas was continuously diffused into the VMS (D5) to provide saturated conditions at that operating temperature for approximately 15 minutes, and thereafter continued throughout the entire cleaning operation. A UV light source comprising a Model 250B Pulsed Xenon UV System, available from Xenon Corporation, Franklin, Mass. with a light pipe connected to the Xenon flash lamp source and plumbed directed into the ozonated VMS (D5) treatment fluid.

Varnish build-up within diesel fuel injector nozzles is a result of free-radical chain reactions within the diesel fuel generated by hot combustion processes. These reactions comprise chain initiation, propagation, branching, and termination steps, all of which produce successive layers of resinous fuel deposits over time that eventually constrain fuel flow. Such contamination must be removed from the nozzles periodically to return them to as-new fuel flow condition. Strong solvents with ultrasonics must be used to dissolve the varnish due to its nature and complexity—a highly cross-linked, plastic-like and gummy surface coating. Acidic cleaner are not useful for this application as they will corrode the carbon steel along with the contaminant film.

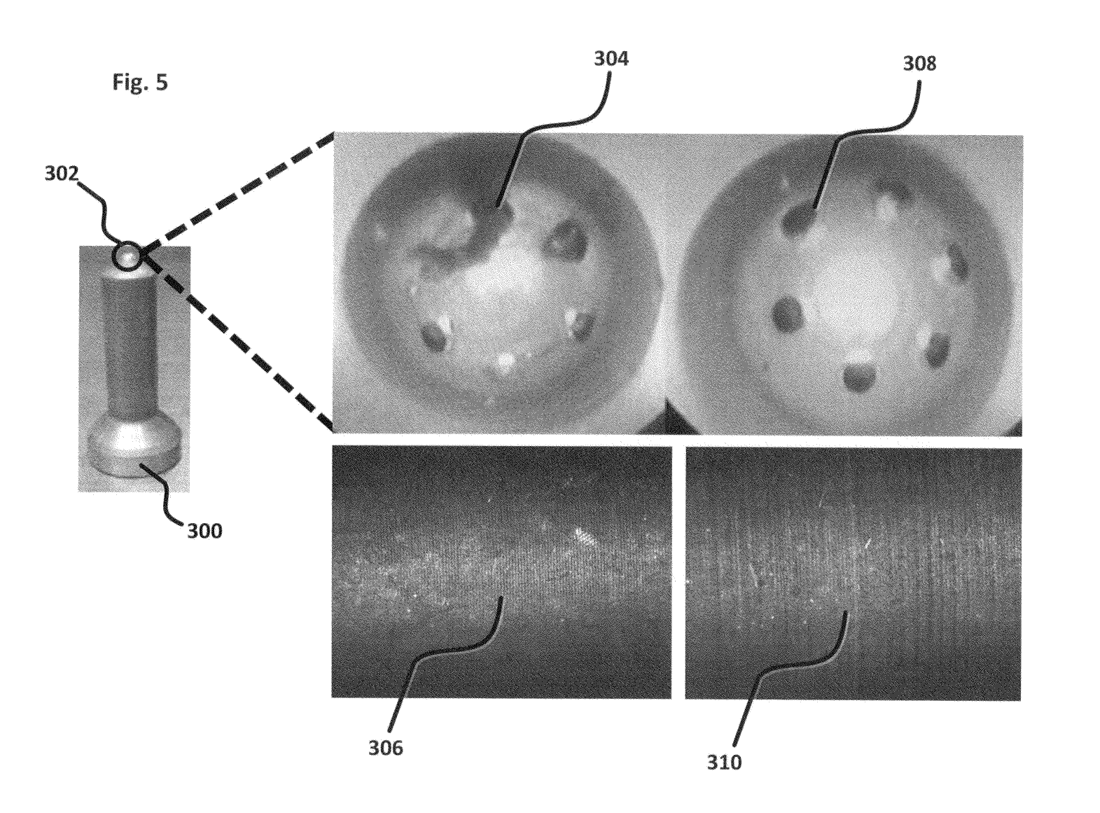

Referring to FIG. 5, a plugged fuel injector nozzle (300) contains a multi-ported nozzle tip (302) which over time builds up with and subsequently clogs with an organic resin deposit termed varnish. Under magnification, varnish appears as a uniform reddish film (306) covering the entire surface of the fuel injector tip (302) and can become extremely gummy and thick within and surrounding the fuel injection ports (304).

The present invention was used to clean and salvage clogged diesel fuel injector nozzles. A small batch of fuel injector nozzles was placed in a stainless steel mesh basket and subjected the exemplary treatment method of FIG. 2, described briefly as follows:
1. Pre-soaked dirty fuel injectors for 5 minutes in ozone-saturated VMS (D5) at a temperature of 40 degrees C.
2. Exposed dirty fuel injectors to continuous ultrasonic treatment for 5 minutes at 40 kHz, and while radiating the treatment fluid and substrates with pulsed Xenon light having significant UV spectral output in the wavelength range between 200 nm and 400 nm. The Xenon pulsed flash lamp was energized every minute for 15 seconds.
3. Repeated step 1 and step 2 for a total of three soak-wash cycles, for a total treatment time of 30 minutes: 15 minutes soaking in ozonated VMS (D5), 15 minutes of ultrasonic agitation, and approximately 4 minutes of pulsed UV light exposure.
4. Lifted treated fuel injectors from treatment bath and drained excess ozonated VMS (D5) back into bath.
5. Rinsed treated fuel injectors and recovered VMS (D5) using centrifugal liquid CO₂ processing.

Again referring to FIG. 5, following treatment using the present invention the fuel injector nozzle ports (308) are clean and free of resinous deposits. Moreover, under magnification the treated and liquid CO₂ rinsed surface (310) appears much cleaner with the reddish varnish removed—evidenced by the re-emergence of the deeper machining grooves previously covered by the thick reddish resinous contaminating film.

Other applications contemplated for the present invention include, but are not limited to:

1. Cleaning and disinfection of hospital and medical substrates such as bedding and endoscopes.
2. Cleaning and disinfection of sports equipment such as protective pads.
3. Cleaning and disinfection of carpets.
4. Cleaning and disinfection of clothing and uniforms.
5. Cleaning and disinfection of pharmaceutical equipment.

There is presented a method and apparatus for removing contaminants from a substrate surface comprising:
a. Contacting the substrate surface, which has said contaminants, with a volatile methyl siloxanes (VMS) treatment fluid, containing a dissolved ozone (O₃) gas;
b. Applying ultraviolet (UV) light to said treatment fluid;
Whereby said method removes or denatures said contaminants from the substrate surface.

The method and apparatus further having said contaminants are biological endotoxin (BET), biological, organic or inorganic; the dissolved ozone (O₃) gas concentration range is from 5 to 5000 parts per million; a temperature range is −20 degree Celsius to +100 degree Celsius; range of the UV light is between 200 nm and 400 nm; the UV light is derived from a pulsed Xenon light source; the substrate surface is immersed into the volatile methyl siloxanes (VMS) treatment fluid, which contains the dissolved ozone (O₃) gas; the volatile methyl siloxanes (VMS) treatment fluid, which contains the dissolved ozone (O₃) gas, is sprayed onto the substrate surface; the substrate surface is mounted upon a spin processor; the substrate surface is further treated with ultrasonic energy, heat, vacuum, or carbon dioxide cleaning; the concentration of the dissolved ozone (O₃) gas is monitored and controlled using a spectrophotometer and an ozone generator.

As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting; but rather, to provide an understandable description of the invention.

The terms “a” or “an”, as used herein, are defined as: one or more than one. The term plurality, as used herein, is defined as: two or more than two. The term another, as used herein, is defined as at least a second or more. The terms including and/or having, as used herein, are defined as comprising (i.e., open language). The term coupled, as used herein, is defined as connected, although not necessarily directly, and not necessarily mechanically.

Any element in a claim that does not explicitly state “means for” performing a specific function, or “step for” performing a specific function, is not be interpreted as a “means” or “step” clause as specified in 35 U.S.C. Sec. 112, Paragraph 6. In particular, the use of “step of” in the claims herein is not intended to invoke the provisions of 35 U.S.C. Sec. 112, Paragraph 6. All cited and referenced patents, patent applications and literature are all incorporated by reference in entirety.

I claim:

1. A method for removing contaminants from a substrate surface comprising:
a. Contacting the substrate surface, which has said contaminants, with a volatile methyl siloxanes (VMS) treatment fluid, containing a dissolved ozone (O₃) gas;
b. Applying ultraviolet (UV) light to said treatment fluid;
Whereby said method removes or denatures said contaminants from the substrate surface.

2. The method of claim 1, wherein said contaminants are biological endotoxin (BET), biological, organic or inorganic.

3. The method of claim 1, wherein a range of the dissolved ozone (O₃) gas concentration is from 5 to 5000 parts per million.

4. The method of claim 1, wherein a temperature range is −20 degree Celsius to +100 degree Celsius.

5. The method of claim 1, wherein a range of the UV light is between 200 nm and 400 nm.

6. The method of claim 1, wherein the UV light is derived from a pulsed Xenon light source.

7. The method of claim 1, wherein the substrate surface is immersed into the volatile methyl siloxanes (VMS) treatment fluid, which contains the dissolved ozone (O₃) gas.

8. The method of claim 1, wherein the volatile methyl siloxanes (VMS) treatment fluid, which contains the dissolved ozone (O₃) gas, is sprayed onto the substrate surface.

9. The method of claim 7, wherein the substrate surface is mounted upon a spin processor.

10. The method of claim 1, wherein the substrate surface is further treated with ultrasonic energy, heat, vacuum, or carbon dioxide cleaning.

11. The method of claim 1, wherein concentration of the dissolved ozone (O₃) gas is monitored and controlled using a spectrophotometer and an ozone generator.
